(19) United States
(12) Patent Application Publication
(10) Pub. No.: US 2023/0317220 A1
(43) Pub. Date: Oct. 5, 2023

(54) SYSTEMS AND METHODS FOR USING PERSON CONTROLLED IDENTIFIERS

(71) Applicant: Change Healthcare Holdings, LLC, Nashville, TN (US)

(72) Inventors: Suprigya Babu, Redmond, WA (US); Arien Malec, Oakland, CA (US); Jessica Jones, Seattle, WA (US); Yogesh Sinai Kakodkar, Kenmore, WA (US); Rahul Patil, Nashville, TN (US); Theodore W Decastri, JR., Chapmansboro, TN (US)

(21) Appl. No.: 17/710,368

(22) Filed: Mar. 31, 2022

(51) Int. Cl. G16H 10/60; G06Q 40/08; G06Q 20/02

(52) U.S. Cl. CPC G16H 10/60; G06Q 40/08; G06Q 20/023

(57) ABSTRACT

In an embodiment, systems and methods for generating and using person-controlled identifiers are provided. Coverage data for patients, received from multiple payors or collected from submitted claims, is analyzed to identify patients across payors that are the same patient. A person-controlled identifier is associated with each patient and is linked to the coverage data for the patient for each payor. Accordingly, the person-controlled identifier may uniquely identify the patient and their associated coverage at each payor. In the event that the patient changes payors, the person-controlled identifier may be linked to the new payor. Later, when the patient receives medical care from a medical provider, the patient may provide the person-controlled identifier to the medical provider instead of their payor issued card or number. The medical provider may then use the person-controlled identifier to determine the payor associated with the patient and to determine eligibility.

### BACKGROUND

In healthcare, there is not a single identifier that can be used to identify a patient across multiple payor systems. Generally, when a patient gets a medical service from a medical provider, the patient provides a card with identification information that uniquely identifies them to a particular payor. The medical provider may then submit a request for reimbursement to a particular payor through one or more claims clearinghouses. The request may include the identification information.

For various reasons, the payor associated with a patient may change. For example, the employer of the patient may change insurance payors to save money. However, the patient may not be aware of the change (or may just forget) and present an old card to a medical provider for a medical service. When the medical provider verifies the insurance coverage with the payor, the payor may reject the claim because the patient is no longer covered by the payor. However, because there is no universal identifier, there is no easy way for the medical provider to discover the new insurance payor associated with the patient.

### SUMMARY

In an embodiment, systems and methods for generating and using person-controlled identifiers are provided. Coverage data for patients, received from multiple payors or collected from submitted claims, is analyzed to identify patients across multiple payors. A person-controlled identifier is associated with each patient and is linked to the coverage data for the patient for each relevant payor. Accordingly, the person-controlled identifier may uniquely identify the patient and his/her associated coverage at each associated payor. In the event that the patient changes payors, the person-controlled identifier may be linked to the new payor. Later, when the patient receives medical care from a medical provider, the patient may provide the person-controlled identifier to the medical provider instead of his/her payor-issued cards or numbers. The medical provider may then use the person-controlled identifier to determine the payor or payors associated with the patient and to determine the eligibility of the patient.

The systems and methods described herein provide the following advantages. The person-controlled identifier is uniquely associated with a patient, and may never change, even when the patient changes insurance providers or payors. This frees the patient from having to remember who his/her payor is and from carrying multiple, and possibly outdated, insurance cards. In addition, insurance payors may no longer have to print and mail new insurance cards to patients every year.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a person-controlled identifier system and method. Together with the description, the figures further serve to explain the principles of the person-controlled identifier system and method described herein and thereby enable a person skilled in the pertinent art to make and use the person-controlled identifier system and method.

FIG. 1 is an example environment for using person-controlled identifiers (PCIDs);

FIG. 2 is an illustration of an example PCID table generated from three patient coverage tables;

FIG. 3 is an illustration of an example method for providing insurance coverage data to a medical provider based on a PCID associated with a patient;

FIG. 4 is an illustration of an example method for providing insurance coverage data to a medical provider based on a name and demographic information associated with a patient; and

FIG. 5 shows an example computing environment in which example embodiments and aspects may be implemented.

### DETAILED DESCRIPTION

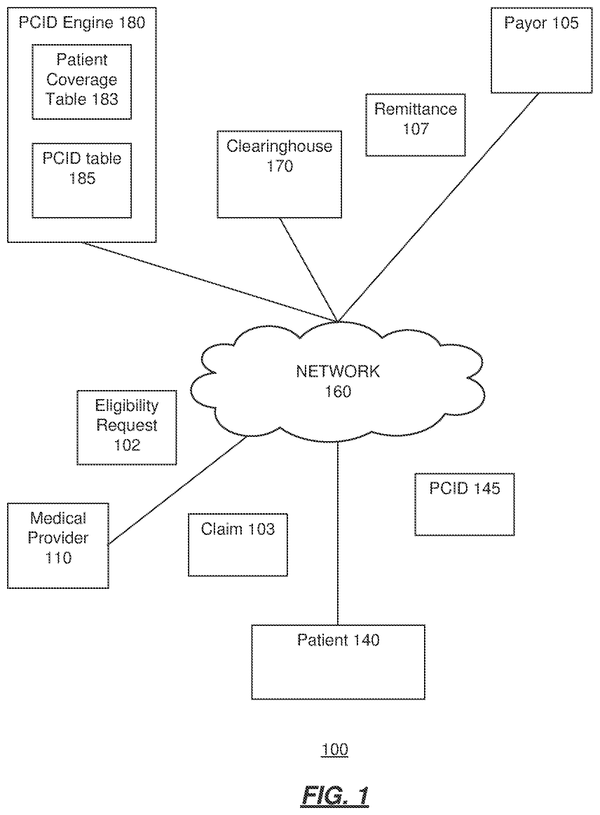

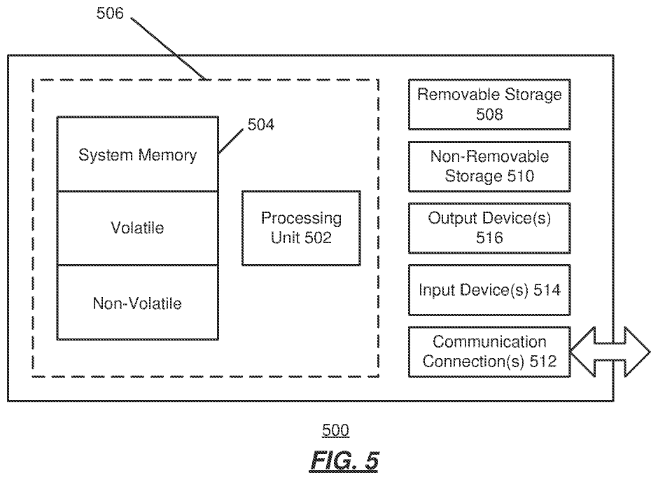

FIG. 1 is an example environment 100 for using person-controlled identifiers (PCIDs) 145. As shown, the environment 100 may include a clearinghouse 170, one or more medical providers 110, one or more payors 105 and one or more patients 140 in communication through a network 160. The network 160 may include a combination of private networks (e.g., LANs) and public networks (e.g., the Internet). Each of the clearinghouse 170, the medical provider 110, the patient 140, and the payor 105 may use, or may be partially implemented by, one or more general purpose computing devices such as the computing device 500 illustrated in FIG. 5.

The clearinghouse 170 may be a medical claims clearinghouse 170 and may receive claims 103 for medical services rendered by medical providers 110 to patients 140. The clearinghouse 170 may then submit each received claim 103 to a corresponding payor 105 (e.g., insurance company or government entity), and may receive remittances 107, or claim payment decisions (e.g., denied, accepted, accepted at some level) from the payors 105 for the claims 103. The clearinghouse 170 may further facilitate transfer of the remittances 107 to the medical providers 110.

The clearinghouse 170 may further receive one or more eligibility requests 102 from medical providers 110 for patients 140. The eligibility request 102 (also referred to in the insurance industry as an Eligibility and Benefit Inquiry 270) is a transaction to inquire about an eligibility and benefits associated with a patient 140. An eligibility request 102 typically includes an identifier or account number issued by a payor 105 for a patient 140.

As described above, one drawback associated with current health systems is the lack of universal identifiers for patients 140 that can be used in eligibility requests 102 and to determine insurance coverage for a patient 140 across multiple payors 105. Currently, each patient 140 may have a different identifier or account number for each payor 105, through which he/she has medical coverage. This may cause difficulty for the patient 140 and medical providers 110 when a patient 140 changes insurance payors 105 but forgets to inform his/her medical provider 110. In such scenarios there is currently no easy way for the medical provider 110 to correctly determine which payor 105 with whom the patient 140 actually has an account.

Accordingly, to solve this problem, the environment 100 may further include a PCID engine 180 that assigns PCIDs 145 to each patient 140. The PCID engine 180 may maintain a PCID table 185 that maps patients 140, through their associated PCIDs 145, to their accounts with one or more of the payors 105. The PCIDs 145 may then be provided to each patient 140 in place of, or in addition to, the identifier or card provided to them by their associated payors 105.

As will be described further below, when a patient 140 goes to a medical provider 110 for medical services, the patient 140 may present his/her PCID 145 to the medical provider 110 instead of the actual card provided by the payor 105 who provides insurance coverage to the patient 140. The medical provider 110 may then use the PCID 145 to request coverage information from the PCID engine 180, which will use the PCID table 185 to determine which payor 105 should receive the eligibility request 102 for the patient 140. In the event that the payor 105 associated with the patient 140 should change, the PCID table 185 is automatically updated by the PCID engine 180.

Note that while the PCID engine 180 is shown as being separate from the clearinghouse 170 and other entities of the environment 100, it is for illustrative purposes only. Depending on the embodiment, the PCID engine 180 may be implemented by one or more of the clearinghouse 170, the payor 105, or the medical provider 110. The PCID engine 180 may be implemented using one or more general purpose computing devices such as the computing device 500 illustrated in FIG. 5.

In some embodiments, the PCID engine 180 may generate the PCID 145 using patient coverage data received from each payor 105. Each payor 105 may provide patient coverage data that identifies each patient 140 that is covered by the payor 105 and the insurance coverage provided by the payor 105 to the patient 140. The patient coverage data received from a payor 105 may be stored by the PCID engine 180 as a patient coverage data table 183. Other data structures may be used.

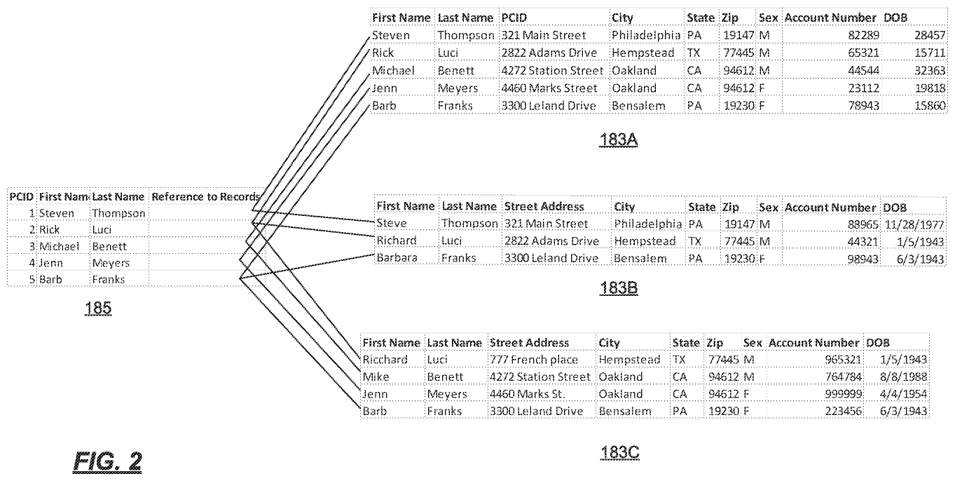

The patient coverage table 183 for a payor 105 may include a record for each covered patient 140. Each record may further include information such as a first and last name of the patient 140, demographic information about the patient 140, an address of the patient 140, and information about the particular insurance policy that covers the patient 140. Other information may be included in each record. Example patient coverage tables 183 are illustrated in FIG. 2.

In some embodiments, rather than receive the patient coverage table data for the patient coverage table 183 from each payor 105, the PCID engine 180 may construct the data based on claims 103 and/or eligibility requests 102 received from medical providers 110. Other information such as coordination of benefits information may be used to construct the data. For example, each submitted claim 103 or eligibility request 102 may include information about the associated patient 140, such as name and policy number as well as certain demographic information. This information may be used by the PCID engine 180 to construct the patient coverage table 183 for each payor 105. The information in the patient coverage table 183 may be constructed or updated periodically (e.g., once a week or month), or every time a new claim 103 or eligibility request 102 is received.

The PCID engine 180 may generate the PCID table 185 from the data in the patient coverage tables 183 for each payor 105. The PCID table 185 may be a mapping of PCIDs 145 to records in some or all of the patient coverage tables 183.

To generate the PCID table 185 the PCID engine 180 may first determine, across all patient coverage tables 183, records of the tables 183 that likely refer to the same patient 140. Depending on the embodiment, the PCID engine 180 may identify records of the tables 183 that likely refer to the same patient 140 based on information from each record such as the name, address, and demographic information.

The PCID engine 180 may identify records that refer to the same patient 140 using a similarity function that accepts as an input two records and outputs a probability that the records refer to the same patient 140. Records with similarities that are above a threshold may be determined to refer to same patient 140. The similarity function may include edit distance functions and Jaro-Wilnkler distance functions, for example. Other methods may be used including training a machine learning model to identify similar records, or by using name, address, or demographic based-matching.

As may be appreciated, because the data in each record for the same patient 140 may vary between patient coverage tables 183, it may be difficult for the PCID engine 180 to determine with a 100% probability that two records refer to the same patient 140. For example, the records for a patient 140 may use different name variations (e.g., Mike vs Michael), may have different addresses due to patients 140 moving, or may include one or more typos.

The PCID engine 180 may identify groups of records from the patient coverage tables 183 that refer to the same patient 140 and may assign each identified group and patient 140 a PCID 145. The PCID engine 180 may then make a record in the PCID table 185 for each assigned PCID 145. Each record of the PCID table 185 may include a pointer or reference to each record of the group of records from the patient coverage tables 183 that were found by the PCID engine 180 to refer to the same patient 140. Alternatively, instead of a reference to each record of the patient coverage table table 183, the PCID table 185 may include the contents of each record.

Each record in the PCID table 185 may also include information about the associated patient 140. This information may include the name of the patient 140, and demographic information about the patient 140. Other information may be included.

As an example, FIG. 2 is an illustration of an example PCID table 185 generated from three patient coverage tables 183 (i.e., the tables 183A, 183B, and 183C). The PCID engine 180 may have processed the records of each table 183 and may have determined five groups of records from the tables 183 that refer to the same patients 140. The PCID engine 180 may have created an entry in the PCID table 185 for each group and assigned each group a PCID 145.

In the example shown, the PCID engine 180 has determined a group of records corresponding to the patient 140 “Steven Thompson” and assigned the group the PCID 145 of 1. The PCID engine 180 has determined a group of records corresponding to the patient 140 “Rick Luci” and assigned the group the PCID 145 of 2. The PCID engine 180 has determined a group of records corresponding to the patient 140 “Michael Bennett” and assigned the group the PCID 145 of 3. The PCID engine 180 has determined a group of records corresponding to the patient 140 “Jenn Meyers” and assigned the group the PCID 145 of 4. The PCID engine 180 has determined a group of records corresponding to the patient 140 “Barb Franks” and assigned the group the PCID 145 of 5.

Each record of the PCID table 185 includes a pointer to each record of the group of records of the patient coverage tables 183 that are associated with the same patient 140 and PCID 145. In the example shown in FIG. 2, the pointers are represented as lines connecting the records of the PCID table 185 to each of the corresponding records in the patient coverage tables 183A, 183B, and 183C.

In some embodiments, once the PCIDs 145 have been created in the PCID table 185, some or all of the PCIDs 145 may be distributed to the corresponding patients 140. The PCIDs 145 may be distributed electronically or may be printed onto a card or other media and physically provided to each patient 140.

In some embodiments, rather than automatically create a PCID 145 for a patient 140, the patient 140 may request a PCID 145. For example, the PCID engine 180 may provide a graphical user interface through which the patient 140 may request a PCID 145. The patient 140 may provide his/her name and other demographic information to the PCID engine 180. In addition, if known, the patient 140 may identify an associated payor 105 and any account numbers associated with the payor 105.

The PCID engine 180 may then use the provided information to find accounts in the various patient coverage tables 183 that are likely associated with the patient 140. The PCID engine 180 may then create a record in the PCID table 185 that points to the records associated with the accounts in the patient coverage table 183 and may provide the corresponding PCID 145 to the patient 140.

The PCID engine 180 may periodically update the PCID table 185 based on changes to the patient coverage tables 183. As may be appreciated, the patient coverage tables 183 may change as patients 140 change insurance plans. Accordingly, when the PCID engine 180 detects that a patient coverage table 183 has changed, it may use the PCID table 185 to determine if the change affects any patient 140 in the PCID table 185, and if so, may update the PCID table 185 to reflect the change.

Continuing the example from FIG. 2, if the record corresponding to the patient 140 “Barb Franks” is removed from the table 183C, indicating that Barb no longer has coverage through the payor 105 associated with the table 183C, the PCID engine 180 may either detect the removal or may receive a notification from the associated payor 105. In response, the PCID engine 180 may update the PCID table 185 by removing the pointer to the table 183C from the record associated with Barb in the PCID table 185.

When a patient 140 goes to a medical provider 110 for a medical service, the patient 140 may present his/her PCID 145 to the medical provider 110. The medical provider 140 may then send the PCID 145 to the PCID engine 180 to determine what insurance coverage is associated with the patient 140. The PCID engine 180 may then determine the insurance coverage associated with the patient 140 using the PCID 145 and the PCID table 185 and may provide information associated with the insurance coverage to the medical provider 110. The medical provider 110 may then use the information associated with the insurance coverage (e.g., account number or identifier) when submitting an eligibility request 102 for the patient 140.

Alternatively, rather than use the PCID 145 to determine the insurance coverage information associated with the patient 140, after a medical service has been performed for the patient 140, the medical provider 110 may submit a claim 103 to the clearinghouse 170 that includes the PCID 145. The clearinghouse 170 may use the PCID 145 to determine the insurance information associated with the patient 140 including payor 105. The clearinghouse may then forward the claim 103 to the relevant payor 105.

The PCID engine 180 may also be used to determine insurance coverage information for a patient 140 using information such as name, address, and demographic information, even when the patient 140 does not have or does not know his/her PCID 145. In such cases, the name, address, and demographic information may be used to either identify a record in the PCID table 185 that matches the patient 140, or in cases where no PCID 145 exists, to identify records in any of the patient coverage tables 183 that likely correspond to the patient 140. Such a feature may be useful in emergency rooms where patients 140 may arrive unconscious, unprepared, or otherwise unable to present their PCID 145 or other insurance coverage information that could be used in an eligibility request 102.

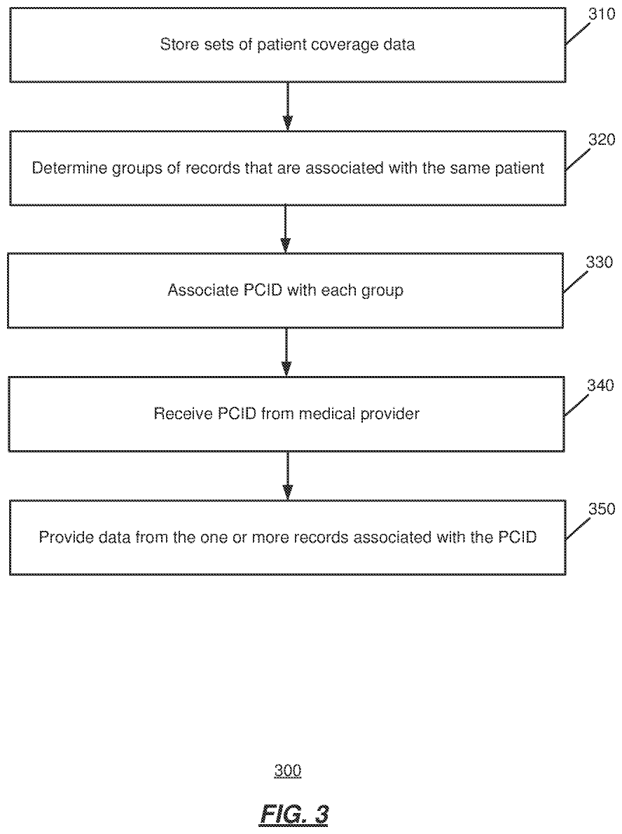

FIG. 3 is an illustration of an example method for providing insurance coverage data to a medical provider based on a PCID associated with a patient. The method 300 may be implemented by a PCID engine 180.

At 310, sets of patient coverage data are stored. The patient coverage data may be stored by the PCID engine 180 as the patient coverage tables 183. In some embodiments, each payor 105 may be associated with its own patient coverage table 183. The patient coverage table 183 for a payor 105 may include a record for each covered patient 140 of the payor 105. Each record may include a name of the patient 140, an account number, an address, and demographic information about the patient 140. The patient coverage data may be provided by the payors 105 to the PCID engine 180 or may be compiled by the PCID engine 180 for each payor 105 based on the claims 103 and eligibility requests 102 received by one or more clearinghouses 170.

At 320, groups of records that are associated with the same patient are determined. The groups of records may be determined by the PCID engine 180. Each record in a group of records may refer to the same patient 140. In some embodiments, each group of records may include a record from some or all of the patient coverage tables 183. The PCID engine 180 may determine a group of records by comparing each record from each patient coverage table 183 using similarity functions and determining those records that are similar and therefore may refer to the same patient 140. The similarity function may consider information from each record such as the name of the patient 140, address of the patient 140, and other demographic information associated with the patient 140 such as age and sex, for example.

At 330, a PCID is associated with each group of records. The PCID 145 may be associated with each group of records by the PCID engine 180. Each PCID 145 may be added to a record of a PCID table 185. Each record of the PCID table 185 may include a PCID 145 and a reference or pointer to each record in the associated group of records spread across the patient coverage tables 183 associated with each payor 105. Each record may further include information taken from the records of the groups of records from the patient coverage tables 183 such as name, address, and demographic information.

At 340, a PCID associated with a first patient is received. The PCID 145 may be received from a medical provider 110. For example, the first patient 140 may have presented the PCID 145 when seeking medical services from the medical provider 110. The medical provider 110 may provide the information to the PCID engine 180 as part of an eligibility request 102.

At 350, data from one or more records of the group of records associated with the received PCID is provided. The data may be provided by the PCID engine 180 to the medical provider 110. The data may be from the records of the patient coverage tables 183 referenced by the record of the PCID table 185 that corresponds to the received PCID 145 of the first patient. The data may be sufficient for the medical provider 110 to generate and send an eligibility request 102 for the patient 140.

In some embodiments, rather than provide the data from the one or more records to the provider 110, the PCID engine 180 may instead provide a link or reference to the one or more records to the medical provider 110. The medical provider 110 may then retrieve the data from the PCID table 185 and/or determine eligibility of the first patient using the link or reference.

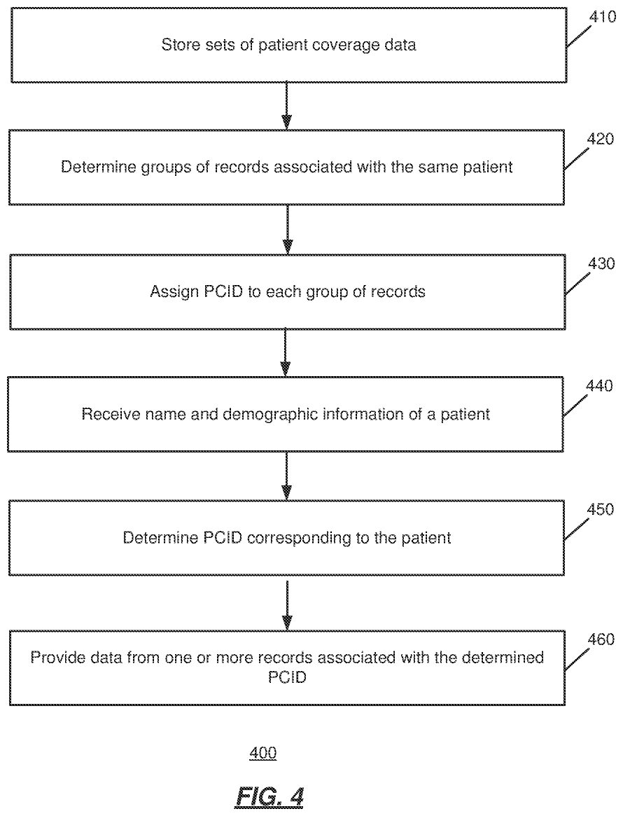

FIG. 4 is an illustration of an example method 400 for providing insurance coverage data to a medical provider based on a name and demographic information associated with a patient. The method 400 may be implemented by a PCID engine 180.

At 410, sets of patient coverage data are stored. The patient coverage data may be stored by the PCID engine 180 as the patient coverage tables 183. In some embodiments, each payor 105 may be associated with its own patient coverage table 183. The patient coverage table 183 for a payor 105 may include a record for each covered patient 140 of the payor 105. Each record may include a name of the patient 140, an account number, an address, and demographic information about the patient 140. The patient coverage data may be provided by the payors 105 to the PCID engine 180 or may be compiled by the PCID engine 180 for each payor 105 based on the claims 103 and eligibility requests 102 received by one or more clearinghouses 170.

At 420, groups of records that are associated with the same patient are determined. The groups of records may be determined by the PCID engine 180. Each record in a group of records may refer to the same patient 140. In some embodiments, each group of records may include a record from some or all of the patient coverage tables 183. The PCID engine 180 may determine a group of records by comparing each record from each patient coverage table 183 using similarity functions and determining those records that are similar and therefore may refer to the same patient 140. The similarity function may consider information from each record such as the name of the patient 140, address of the patient 140, and other demographic information associated with the patient 140 such as age and sex, for example.

At 430, a PCID is assigned to each group of records. The PCID 145 may be assigned to each group of records by the PCID engine 180. Each PCID 145 may be added to a record of a PCID table 185. Each record of the PCID table 185 may include a PCID and a reference or pointer to each record in the associated group of records spread across the patient coverage tables 183 associated with each payor 105. Each record may further include information taken from the records of the groups of records from the patient coverage tables 183 such as name, address, and demographic information.

At 440, name and demographic information are received for a patient. The name and demographic information may be received from a medical provider 110. For example, a patient 140 may not know their insurance coverage information or even their PCID 145. Accordingly, the patient 140 may provide their name and demographic information to the medical provider 140 and the medical provider 110 may provide the information to the PCID engine 180 as part of an eligibility request 102, for example.

At 450, a PCID corresponding to the patient is determined. The PCID 145 of the patient 140 may be determined by the PCID engine 180. In some embodiments, the PCID 145 may be determined using the name and demographic information associated with the patient 140. For example, the PCID engine 180 may look for a record in the PCID table 185 with a name and demographic information that matches the provided name and demographic information of the patient 140. Alternatively, the PCID engine 180 may look for records in the patient coverage tables 183 with a name and demographic information that matches the provided name and demographic information of the patient 140.

At 460, data from one or more records of the group of records associated with the determined PCID is provided. The data may be provided by the PCID engine 180 to the medical provider 110 in response to the eligibility request 102. The data may be from the records of the patient coverage tables 183 referenced by the record of the PCID table 185 that corresponds to the determined PCID 145. The data may include insurance coverage information associated with the patient 140 and may indicate the payor(s)105 associated with the insurance coverage information. The medical provider 110 may use the data to generate and send an eligibility request 102 for the patient.

FIG. 5 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an example system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 408 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
storing sets of patient coverage data from a plurality of payors by a computing device, wherein the set of patient coverage data for a payor comprises a plurality of records, and each record is associated with a patient of a plurality of patients;
determining groups of records from the sets of patient coverage data by the computing device, wherein each record in a group of records is associated with a same patient of the plurality of patients, and wherein each record of a group of records comes from a different set of patient coverage data;
for each group of records from the determined groups of records, associating a person-controlled identifier to the group of records by the computing device;
receiving a person-controlled identifier from a medical provider by the computing device; and
in response to receiving the person-controlled identifier, providing data from one or more records from the group of records that the person-controlled identifier is associated with to the medical provider by the computing device.

2. The method of claim 1, further comprising:
receiving demographic information for a patient of the plurality of patients from a medical provider;
based on the received demographic information for the patient of the plurality of patients, determining a person-controlled identifier associated with the patient; and
providing data from one or more records from the group of records that the determined person-controlled identifier is associated with to the medical provider.

3. The method of claim 1, wherein the data from the one or more records comprises eligibility information or may be used to generate an eligibility request.

4. The method of claim 1, wherein the set of patient coverage data for a payor is provided by the payor.

5. The method of claim 1, wherein the set of patient coverage data for a payor is collected from claims and eligibility requests received by a healthcare clearinghouse.

6. The method of claim 1, wherein the computing device is associated with a healthcare clearinghouse.

7. The method of claim 1, wherein the payors of the plurality of payors are health insurance payors.

8. A system comprising:
at least one processor; and
a computer-readable medium storing computer executable instructions that when executed by the at least one processor cause the at least one processor to:
store sets of patient coverage data from a plurality of payors, wherein the set of patient coverage data for a payor comprises a plurality of records, and each record is associated with a patient of a plurality of patients;
determine groups of records from the sets of patient coverage data wherein each record in a group of records is associated with a same patient of the plurality of patients, and wherein each record of a group of records comes from a different set of patient coverage data;
for each group of records from the determined groups of records, associate a person-controlled identifier to the group of records;
receive a person-controlled identifier from a medical provider; and
in response to receiving the person-controlled identifier, provide data from one or more records from the group of records that the person-controlled identifier is associated with to the medical provider.

9. The system of claim 8, further comprising computer executable instructions that when executed by the at least one processor cause the at least one processor to:
receive demographic information for a patient of the plurality of patients from a medical provider;
based on the received demographic information for the patient of the plurality of patients, determine a person-controlled identifier associated with the patient; and
provide data from one or more records from the group of records that the determined person-controlled identifier is associated with to the medical provider.

10. The system of claim 8, wherein the data from the one or more records comprises eligibility information or may be used to generate an eligibility request.

11. The system of claim 8, wherein the set of patient coverage data for a payor is provided by the payor.

12. The system of claim 8, wherein the set of patient coverage data for a payor is collected from claims and eligibility requests received by a healthcare clearinghouse.

13. The system of claim 8, wherein the system is associated with a healthcare clearinghouse.

14. The system of claim 8, wherein the payors of the plurality of payors are health insurance payors.

15. A non-transitory computer-readable medium storing instructions that when executed by a computing device cause the computing device to:
store sets of patient coverage data from a plurality of payors, wherein the set of patient coverage data for a payor comprises a plurality of records, and each record is associated with a patient of a plurality of patients;
determine groups of records from the sets of patient coverage data wherein each record in a group of records is associated with a same patient of the plurality of patients, and wherein each record of a group of records comes from a different set of patient coverage data;
for each group of records from the determined groups of records, associate a person-controlled identifier to the group of records;
receive a person-controlled identifier from a medical provider; and
in response to receiving the person-controlled identifier, provide data from one or more records from the group of records that the person-controlled identifier is associated with to the medical provider.

16. The non-transitory computer-readable medium of claim 15, further comprising computer executable instructions that when executed by the at least one processor cause the at least one processor to:
receive demographic information for a patient of the plurality of patients from a medical provider;
based on the received demographic information for the patient of the plurality of patients, determine a person-controlled identifier associated with the patient; and
provide data from one or more records from the group of records that the determined person-controlled identifier is associated with to the medical provider.

17. The non-transitory computer-readable medium of claim 15, wherein the data from the one or more records comprises eligibility information.

18. The non-transitory computer-readable medium of claim 15, wherein the set of patient coverage data for a payor is provided by the payor.

19. The non-transitory computer-readable medium of claim 15, wherein the set of patient coverage data for a payor is collected from claims and eligibility requests received by a healthcare clearinghouse.

20. The non-transitory computer-readable medium of claim 15, wherein the system is associated with a healthcare clearinghouse.
